(19) United States
(12) Patent Application Publication
(10) Pub. No.: US 2018/0140243 A1
(43) Pub. Date: May 24, 2018

(54) SYSTEM AND METHOD FOR ATTENTION MANAGEMENT

(71) Applicants: Inventec (Pudong) Technology Corporation, Shanghai (CN); INVENTEC CORPORATION, Taipei City (TW)

(72) Inventor: Ming-Lung WENG, TAIPEI CITY (TW)

(21) Appl. No.: 15/453,845

(22) Filed: Mar. 8, 2017

(30) Foreign Application Priority Data
Nov. 24, 2016 (CN) 201611050447.5

(51) Int. Cl. A61B 5/18; A61B 5/00

(52) U.S. Cl. CPC A61B 5/18; A61B 5/0002

(57) ABSTRACT

The present invention discloses a system and method for attention management, adapted for managing an attention of a transport driver driving a car, and the attention management method comprises the following steps: providing a detection device to configured at the transport driver's forehead to detect a brain wave of the transport driver; using a brain wave algorithm to analyze the brain wave in order to obtain a brain wave analyzed information; determining that the brain wave analyzed information matches an attention deficiency condition or not; and making a warning sound and transmitting a warning message to a remote dispatching center if the brain wave analyzed information matches the attention deficiency condition.

### RELATED APPLICATIONS

This application claims priority to Chinese Application Serial Number 201611050447.5, filed Nov. 24, 2016, which is herein incorporated by reference.

### BACKGROUND

### Field of Invention

The present invention relates to a system and method for attention management.

### Description of Related Art

In the existing technology, there are two ways of avoiding the inattention of transport drivers. The first way is head-down warning method. The head-down warning method detects balance and uses a buzzer to produce a cheap and small ear hanging device. In case the transport driver's head is down, the ear hanging device alarms to remind the transport driver. The disadvantage of the head-down warning method is that the transport driver's head cannot have too much movement, otherwise the ear hanging device alarms continually. However, the transport driver will feel tired if the transport driver's head always keep at the same posture. If the attention of the transport driver is not concentrated or even closes their eyes, as long as the transport driver's head is not down, the head-down warning method is not effective. Accordingly, the head-down warning method cannot warn correctly. The second way is an eyelid detection system. The eyelid detection system takes a picture for the transport driver's face and uses image identification system to detect the transport driver's eyes in order to determine the transport driver dozes or not. If the transport driver wears sunglasses, the camera cannot take the transport driver's eyes, and the eyelid detection system will be not effective. If the transport driver has smaller eyes, the eyelid detection system will misjudge easily. In addition, when the transport driver's attention is not concentrated and be very tired, the eyelid detection system does not alarm if the transport driver still open eyes reluctantly. Therefore, the eyelid detection system cannot eliminate risk early.

### SUMMARY

The present invention provides a system and method for attention management. The present invention uses the detected transport driver's brain wave to determine the physiological status of the transport driver. When the transport driver's attention is not concentrated but not dozes yet, the brain wave detection mechanism of the present invention can discover that the transport driver is at tired status so as to alarm early. In addition, system and method for attention management of the present invention transmits a warning message to a remote dispatching center to let the remote dispatching center realize the mental status instantly.

An aspect of the disclosure is to provide an attention management system. The attention management system is adapted for managing an attention of a transport driver driving a car. The attention management system comprises a detection device and an electronic device. The detection device is configured at the transport driver's forehead to detect a brain wave of the transport driver. The electronic device is configured at the car. The electronic device is connected to the detection device wirelessly. The electronic device uses a brain wave algorithm to analyze the brain wave in order to obtain brain wave analyzed information. The electronic device determines that the brain wave analyzed information matches an attention deficiency condition or not. The electronic device makes a warning sound and transmits a warning message to a remote dispatching center if the brain wave analyzed information matches the attention deficiency condition.

In one embodiment of the present invention, the electronic device comprises: a processing unit, a storage unit, an alarm unit, and a transceiver unit. The processing unit uses the brain wave algorithm to analyze the brain wave in order to obtain the brain wave analyzed information. The storage unit is connected to the processing unit. The storage unit stores the attention deficiency condition. The alarm unit is connected to the processing unit. The processing unit controls the alarm unit to make the warning sound if the processing unit determines that the brain wave analyzes information matches the attention deficiency condition. The display unit is connected to the processing unit. The processing unit controls the display unit to display a warning image if the processing unit determines that the brain wave analyzes information matches the attention deficiency condition. The transceiver unit is connected to the processing unit. The processing unit controls the transceiver unit to transmit the warning message to the remote dispatching center if the processing unit determines that the brain wave analyzes information matches the attention deficiency condition.

In one embodiment of the present invention, the attention deficiency condition is defined as that: in the brain wave analyzed information, an addition result of a first specific brain wave's ratio and a second specific brain wave' ratio is greater than a default ratio. The first specific brain wave has a first frequency band, and the second specific brain wave has a second frequency band.

In one embodiment of the present invention, the processing unit determines that the detection device touches the transport driver's forehead correctly or not according to continuity of the brain wave detected by the detection device before analyzing the brain wave through the brain wave algorithm.

In one embodiment of the present invention, the processing unit determines that the detection device is not touched the transport driver's forehead correctly if the brain wave is not continued. The processing unit controls the alarm unit to make a reminding sound. The processing unit controls the display unit to display a reminding image. The processing unit controls the transceiver unit to transmit a reminding message to the remote dispatching center.

In one embodiment of the present invention, the detection device and the electronic device are connected wirelessly through Bluetooth transmission.

An aspect of the disclosure is to provide an attention management method, adapted for managing an attention of a transport driver driving a car. The attention management method comprises the following steps: providing a detection device to configured at the transport driver's forehead to detect a brain wave of the transport driver; using a brain wave algorithm to analyze the brain wave in order to obtain a brain wave analyzed information; determining that the brain wave analyzed information matches an attention deficiency condition or not; and controlling an alarm unit to make a warning sound and controlling a transceiver unit to transmit a warning message to a remote dispatching center if the brain wave analyzed information matches the attention deficiency condition.

In one embodiment of the present invention, the attention management method further comprises the following step: controlling a display unit to display a warning image if the brain wave analyzed information matches the attention deficiency condition.

In one embodiment of the present invention, the attention deficiency condition is defined as that an addition result of a first specific brain wave's ratio and a second specific brain wave' ratio is greater than a default ratio. The first specific brain wave has a first frequency band, and the second specific brain wave has a second frequency band.

In one embodiment of the present invention, the attention management method further comprises the following steps: determining that the detection device touches the transport driver's forehead correctly or not according to continuity of the brain wave detected by the detection device; determining that the detection device is not touched the transport driver's forehead correctly if the brain wave is not continued; and controlling the alarm unit to make a reminding sound, controlling the display unit to display a reminding image, and controlling the transceiver unit to transmit a reminding message to the remote dispatching center.

It is to be understood that both the foregoing general description and the following detailed description are by examples, and are intended to provide further explanation of the invention as claimed.

### BRIEF DESCRIPTION OF THE DRAWINGS

The invention can be more fully understood by reading the following detailed description of the embodiment, with reference made to the accompanying drawings as follows:

FIG. 1 is a block diagram of the attention management system according to the first embodiment of the present invention; and

FIG. 2 is a flowchart of the attention management method according to the first embodiment of the present invention.

### DETAILED DESCRIPTION

Reference will now be made in detail to the present embodiments of the invention, examples of which are illustrated in the accompanying drawings. Wherever possible, the same reference numbers are used in the drawings and the description to refer to the same or like parts.

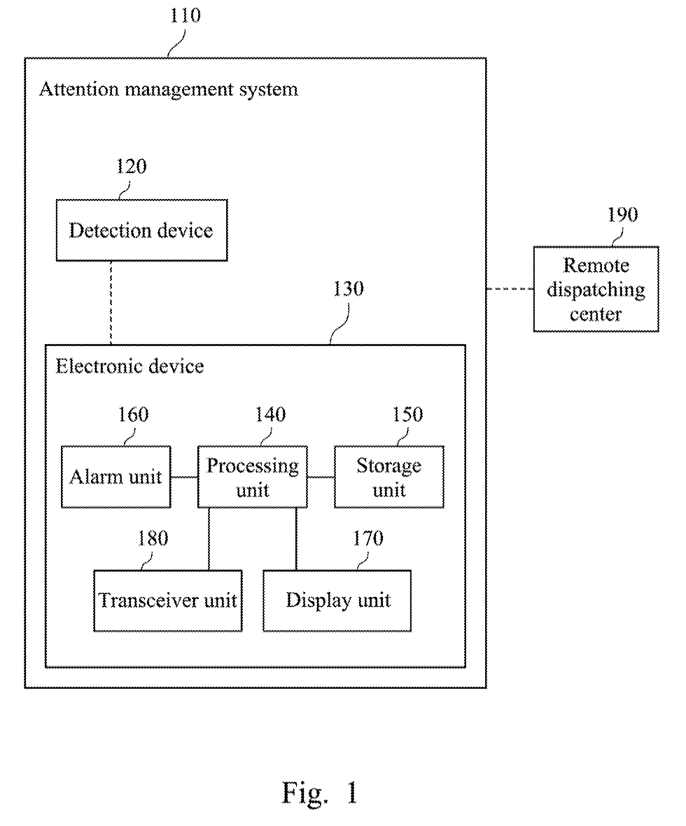

The present invention discloses an attention management system, which is adapted for managing an attention of a transport driver driving a car. The attention management system reminds and alarms when the transport driver's attention is not concentrated. The attention management system also returns the status of the transport driver to a remote dispatching center instantly. FIG. 1 is a block diagram of the attention management system according to the first embodiment of the present invention. The attention management system 110 comprises a detection device 120 and an electronic device 130. The detection device can be a handset detector which detects the brain wave of the transport driver. The detection device 120 is configured at the forehead of the transport driver. The detection device 120 uses simple forehead skin one-point touch electronic detection technique, which is easy to wear so as to the transport driver can wear that by himself. The forehead skin has no hair, so the electrode of the detection device 120 can touch the forehead skin fully, in addition, that can avoid collecting the wrong brain waves.

The electronic device 130 is configured at a car; the detection device 120 is connected wirelessly to the electronic device 130 through the Bluetooth transmission. The electronic device 130 can be a computer system or a smart phone. The electronic device 130 comprises a processing unit 140, a storage unit 150, an alarm 160, a display unit 170, and a transceiver unit 180. The storage unit 150, the alarm 160, the display unit 170, and the transceiver unit 180 are connected to the processing unit 140 respectively. The processing unit can be a central processor of the computer system or the smart phone. The storage unit 150 can be a hard disk or a memory. The alarm unit 160 can be a buzzer. The alarm unit 160 also can be a speaker which is used to make a default warning sound. The display unit 170 can be a monitor. The transceiver unit 180 can be a communication interface.

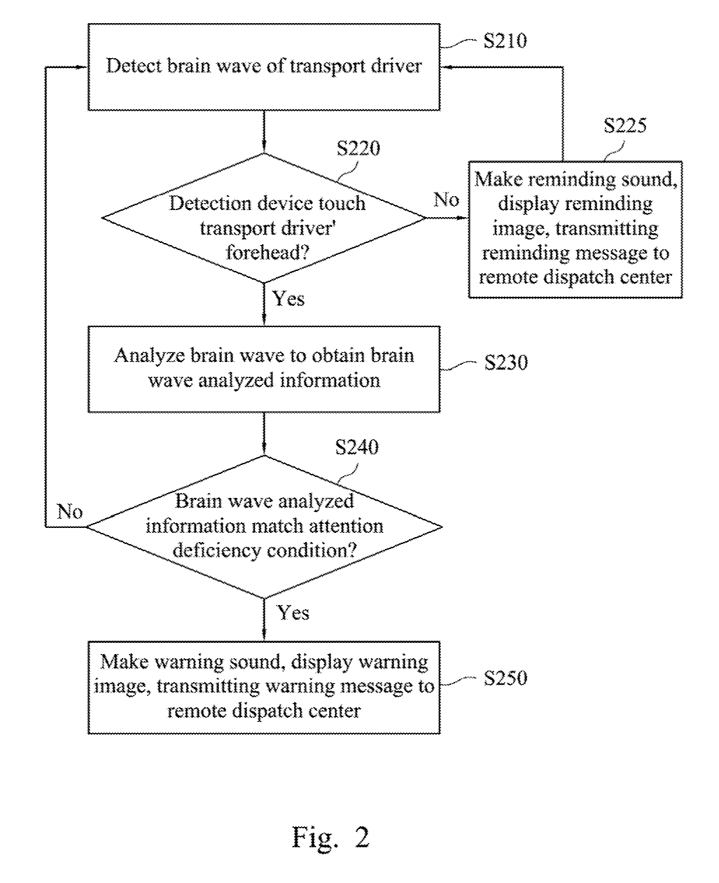

FIG. 2 is a flowchart of the attention management method according to the first embodiment of the present invention. Firstly, the detection device 120 detects brain wave of a transport driver (Step S210). Next, the processing unit 140 determines that the detection device 120 touches the transport driver's forehead correctly or not according to continuity of the brain wave detected by the detection device 120 (Step S220). The processing unit 140 determines that the detection device 120 is not touched the transport driver's forehead correctly if the brain wave is not continued. Then, the processing unit 140 controls the alarm unit 160 to make a reminding sound. The processing unit 140 controls the display unit 170 to display a reminding image. The processing unit 140 controls the transceiver unit 180 to transmit a reminding message to the remote dispatching center 190 (Step S225).

For example, if the processing unit 140 determines that the detection device 120 is not touched to the forehead of the transport driver correctly, the processing unit 140 controls the alarm unit 160 (such like a buzzer) to make a sound with high decibel in order to remind the transport driver. Or, the processing unit 140 controls the alarm unit 160 (such like speaker) to make a default sound “Please wear it again”, so as to remind the transport driver. The processing unit 140 also controls the display unit 170 (such like a monitor) to display a reminding image. The remaining image can be that “Unable to detect, please wear it again.” In addition, the attention management system 110 also transmits a reminding message to the remote dispatching center 190. The remote dispatching center 190 can talk with the transport driver through the radio intercom to request the transport driver wear the detection device 120 again.

If the determination of Step S220 is yes, the processing unit 140 uses a brain wave algorithm to analyze the brain wave in order to obtain a brain wave analyzed information (Step S230). Next, the processing unit 140 determines that the brain wave analyzed information matches an attention deficiency condition or not (Step S240). The definition of the attention deficiency condition is stored at the storage unit 150. The attention deficiency condition is defined as that: in the brain wave analyzed information, an addition result of a first specific brain wave's ratio and a second specific brain wave' ratio is greater than a default ratio. The first specific brain wave has a first frequency band, and the second specific brain wave has a second frequency band. In the embodiment of the present invention, the first specific brain wave is delta (δ) signal at 0.1˜3 Hz, and the second specific brain wave is alpha (α) signal at 8˜9. The default ratio is related to the brain wave algorithm, and the default ration can be set by the producer of the attention management system 110.

In the embodiment of the present invention, the default ratio is 30%. If in the brain wave analyzed information, an addition result of the delta signal's ratio and the alpha signal's ration is greater than 30%, the processing unit 140 controls the alarm unit 160 to make a warning sound. Also, the processing unit 140 controls the display unit 170 to display a warning image, and the processing unit 140 controls the transceiver unit 180 to transmit a warning message to the remote dispatching center 190 (Step 250).

For example, if the processing unit 140 determines that the brain wave analyzed information matches the attention deficiency condition, the processing unit 140 controls the alarm unit 160 (such like a buzzer) to make a sound with high decibel in order to wake the transport driver. Or, the processing unit 140 controls the alarm unit 160 (such like speaker) to make a default sound “Please concentrate”, so as to awake the transport driver. The processing unit 140 also controls the display unit 170 (such like a monitor) to display a warning image. The warning image can be that “Please concentrate.” In addition, the attention management system 110 also transmits a warning message to the remote dispatching center 190. The remote dispatching center 190 can talk with the transport driver through the radio intercom to request the transport driver focus on driving.

If the determination of Step S240 is no, return to the Step S210 and detect the brain wave of the transport driver continually. In other embodiments of the present invention, the detection device 120 detects the brain wave of the transport driver periodically. For example, the detection device 120 detects the brain wave every minute.

The present invention provides a system and method for attention management. The present invention uses the detected transport driver's brain wave to determine the physiological status of the transport driver. When the transport driver's attention is not concentrated but not dozes yet, the brain wave detection mechanism of the present invention can discover that the transport driver is at tired status so as to alarm early. In addition, system and method for attention management of the present invention transmits a warning message to a remote dispatching center to let the remote dispatching center realize the mental status instantly.

Although the present invention has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. An attention management system, adapted for managing an attention of a transport driver driving a car, comprising:
a detection device, configured at the transport driver's forehead to detect a brain wave of the transport driver; and
an electronic device, configured at the car, wherein the electronic device is connected to the detection device wirelessly, the electronic device uses a brain wave algorithm to analyze the brain wave in order to obtain a brain wave analyzed information, the electronic device determines that the brain wave analyzed information matches an attention deficiency condition or not, the electronic device makes a warning sound and transmits a warning message to a remote dispatching center if the brain wave analyzed information matches the attention deficiency condition.

2. The attention management system of claim 1, wherein the electronic device comprises:
a processing unit, using the brain wave algorithm to analyze the brain wave in order to obtain the brain wave analyzed information;
a storage unit, connected to the processing unit, wherein the storage unit stores the attention deficiency condition;
an alarm unit, connected to the processing unit, wherein the processing unit controls the alarm unit to make the warning sound if the processing unit determines that the brain wave analyzes information matches the attention deficiency condition;
a display unit, connected to the processing unit, wherein the processing unit controls the display unit to display a warning image if the processing unit determines that the brain wave analyzes information matches the attention deficiency condition; and
a transceiver unit, connected to the processing unit, wherein the processing unit controls the transceiver unit to transmit the warning message to the remote dispatching center if the processing unit determines that the brain wave analyzes information matches the attention deficiency condition.

3. The attention management system of claim 2, wherein the attention deficiency condition is defined as that: in the brain wave analyzed information, an addition result of a first specific brain wave's ratio and a second specific brain wave' ratio is greater than a default ratio, the first specific brain wave has a first frequency band, and the second specific brain wave has a second frequency band.

4. The attention management system of claim 2, wherein the processing unit determines that the detection device touches the transport driver's forehead correctly or not according to continuity of the brain wave detected by the detection device before analyzing the brain wave through the brain wave algorithm.

5. The attention management system of claim 4, wherein the processing unit determines that the detection device is not touched the transport driver's forehead correctly if the brain wave is not continued, the processing unit controls the alarm unit to make a reminding sound, the processing unit controls the display unit to display a reminding image, the processing unit controls the transceiver unit to transmit a reminding message to the remote dispatching center.

6. The attention management system of claim 1, wherein the detection device and the electronic device are connected wirelessly through Bluetooth transmission.

7. An attention management method, adapted for managing an attention of a transport driver driving a car, comprising:
providing a detection device to configured at the transport driver's forehead to detect a brain wave of the transport driver;
using a brain wave algorithm to analyze the brain wave in order to obtain a brain wave analyzed information;
determining that the brain wave analyzed information matches an attention deficiency condition or not; and
controlling an alarm unit to make a warning sound and controlling a transceiver unit to transmit a warning message to a remote dispatching center if the brain wave analyzed information matches the attention deficiency condition.

8. The attention management method of claim 7, further comprising:
controlling a display unit to display a warning image if the brain wave analyzed information matches the attention deficiency condition.

9. The attention management method of claim 8, wherein the attention deficiency condition is defined as that an addition result of a first specific brain wave's ratio and a second specific brain wave' ratio is greater than a default ratio, the first specific brain wave has a first frequency band, and the second specific brain wave has a second frequency band.

10. The attention management method of claim 8, before analyzing the brain wave through the brain wave algorithm, further comprising:
determining that the detection device touches the transport driver's forehead correctly or not according to continuity of the brain wave detected by the detection device;
determining that the detection device is not touched the transport driver's forehead correctly if the brain wave is not continued; and
controlling the alarm unit to make a reminding sound, controlling the display unit to display a reminding image, and controlling the transceiver unit to transmit a reminding message to the remote dispatching center.
